(19) United States
(12) Patent Application Publication
(10) Pub. No.: US 2026/0224189 A1
(43) Pub. Date: Aug. 6, 2026

(54) LIVE ANATOMICAL MAPPING OF THE LEFT ATRIUM WITH ROBOT-ASSISTED INTRA-CARDIAC ECHOCARDIOGRAPH

(71) Applicant: Siemens Medical Solutions USA, Inc., Malvern, PA (US)

(72) Inventors: Paul Klein, Princeton, NJ (US); Yue Zhang, Short Hills, NJ (US); Athira Jane Jacob, Plainsboro, NJ (US); Young-Ho Kim, West Windsor, NJ (US); Gareth Funka-Lea, Princeton, NJ (US)

(21) Appl. No.: 19/043,622

(22) Filed: Feb. 3, 2025

(51) Int. Cl. A61B 8/08; A61B 8/00; A61B 90/00; G16H 40/63

(52) U.S. Cl. CPC A61B 8/0883; A61B 8/4245; A61B 8/4444; A61B 8/467; A61B 8/5215; A61B 8/543; A61B 90/08; G16H 40/63; A61B 2090/0807; A61B 2576/023

(57) ABSTRACT

Systems and methods for generating an automated overview of the left atrial anatomy for use in live anatomical mapping during a procedure. A robotic drive is used to steer a catheter under ICE image guidance without additional positioning mechanisms. The robotic drive is controlled by an AI based agent, for example that has been trained using deep reinforcement learning (DRL).

### FIELD

This disclosure relates to medical imaging.

### BACKGROUND

Catheter ablation is a minimally invasive procedure that treats abnormal heart rhythms, or arrhythmias, by using heat, cold, or pulsed electric field energy to destroy tissue in the heart. A common treatment option during the ablation procedure is Pulmonary Veins Isolation (PVI) which requires a detailed and accurate visualization of the cardiac anatomy. Quantification of the anatomy has generally been done using cardiac computed tomography (CT) or transesophageal echocardiogram (TEE). However, there are disadvantages with both CT and TEE. CT is a pre-procedure imaging procedure that uses ionizing radiation. TEE requires general anesthesia and the associated risks and the administration of anesthesia requires the presence of anesthesia staff to be present. Recently the use of Intra-cardiac echocardiography (ICE) as an alternative has been proposed as ICE may avoid certain drawbacks associated with CT and TEE. For example, ICE may be performed by an operator with the patient under conscious sedation, without the need for endotracheal intubation. ICE further eliminates the risk of esophageal trauma. Unlike traditional transthoracic or transesophageal echocardiography, which relies on external or semi-invasive approaches, ICE employs a catheter-based probe inserted directly into the heart or nearby vasculature. This proximity to the heart provides high-resolution, real-time images of intracardiac anatomy and physiology, offering beneficial insights for diagnostic and interventional purposes.

Despite these advantages, ICE is not without limitations. ICE requires specialized equipment and expertise to perform safely and efficiently. Such cost considerations and the need for advanced operator training may limit its widespread adoption. In the existing protocol, ICE may be seen as an opportunity for time saving during the ablation procedure, however depending on operator skill and image quality, navigation may be challenging and hinder this benefit. Furthermore, due to navigation constraints, the coverage of the anatomical model may be suboptimal and leave uncertainties in the overall appearance of the anatomy, to be eluded by an ablation procedure.

### SUMMARY

By way of introduction, the preferred embodiments described below include methods, systems, instructions, and computer readable media for live anatomical mapping with robot-assisted intra-cardiac echocardiograph.

In a first aspect, a method for live anatomical mapping of a left atrium with robot assisted intra-cardiac echocardiography (ICE), the method comprising: inserting an ICE catheter into a patient, wherein catheter steering controls of the ICE catheter are controlled at least in part by a machine trained agent; automatically maneuvering the ICE catheter, using instructions from the machine trained agent, from a first position in a patient's anatomy to a second position in the patient's anatomy while acquiring image data of the patient's anatomy; and generating a model of the patient's anatomy from the acquired image data.

In a second aspect, a method for training an agent to operate catheter steering controls of a ICE catheter, the method comprising: acquiring a plurality of pre-operative cardiac CT volumes; generating a simulated cardiac environment from the plurality of pre-operative cardiac CT volume; and training an agent to maneuver the ICE catheter from a first position in the simulated cardiac environment to a second position in the simulated cardiac environment using deep reinforcement learning.

In a third aspect, a system for live anatomical mapping of a left atrium of a patient with robot assisted intra-cardiac echocardiography (ICE), the system comprising: a machine trained agent, the machine trained agent trained using deep reinforcement learning using a simulated volumetric environment; and an ICE catheter comprising steering controls that are controlled at least in part by the machine trained agent; wherein the machine trained agent controls movement of the ICE catheter from a first position to a second position while acquiring imaging data of at least the left atrium.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 depicts an example robotic catheter navigation system according to an embodiment.

FIG. 2 depicts an example of the catheter bending geometry of the example robotic catheter navigation system of FIG. 1.

FIG. 3 depicts an example ultrasound system for autonomous navigation by an ICE catheter according to an embodiment.

FIG. 4 depicts an example method for live anatomical mapping with robot-assisted intra-cardiac echocardiograph according to an embodiment.

FIG. 5 depicts an example training method for training an agent for live anatomical mapping with robot-assisted intra-cardiac echocardiograph.

FIG. 6 depicts an example of deep reinforcement learning.

FIG. 7 depicts an example artificial neural network.

FIG. 8 depicts an example convolutional neural network.

### DETAILED DESCRIPTION

Embodiments described herein provide systems and methods for a fully automated overview of the left atrial anatomy. Embodiments leverage a robotic drive to steer a catheter under ICE image guidance without additional positioning mechanisms. The robotic drive is controlled by an AI based agent, for example that has been trained using deep reinforcement learning (DRL). Image data acquired by the ICE catheter may be used to provide live anatomical mapping during a procedure.

As described herein, examples use ICE and an ablation procedure for a patient although other procedures may be performed using the systems and methods described here. An ablation procedure is a minimally invasive treatment used to correct abnormal heart rhythms (arrhythmias). Ablation procedures involve threading a thin catheter through a vein or artery to the heart under imaging guidance. Once positioned, the catheter delivers energy to targeted areas of heart tissue causing the arrhythmia. This energy creates small, controlled scars that disrupt faulty electrical pathways, restoring normal rhythm. Intra-cardiac echocardiography (ICE) as described above utilizes ultrasound waves to generate images. An ICE probe typically consists of a phased array transducer mounted on a flexible catheter. The catheter is introduced into the venous system of a patient, commonly through the femoral or internal jugular vein, and advanced into the heart under fluoroscopic or ultrasound guidance. Once positioned, the probe captures imaging data that can be reconstructed into 3D images using image processing algorithms. These images may be rotated, magnified, and viewed from various angles, offering a dynamic understanding of intracardiac structures.

One of the key advantages of ICE is its ability to provide real-time guidance during minimally invasive cardiac procedures. For example, during catheter-based interventions for arrhythmias such as atrial fibrillation, ICE facilitates precise catheter positioning and visualization of ablation targets. Similarly, in structural heart interventions, ICE helps delineate anatomical landmarks and verify device placement. By providing direct intracardiac visualization, ICE eliminates the need for external imaging modalities like fluoroscopy in certain cases, thereby reducing radiation exposure to both the patient and the medical team.

The ICE catheter is typically controlled using a steering control operated by a trained clinician. The catheter is typically a thin, flexible tube equipped with an ultrasound transducer at its tip, which can emit and receive sound waves to visualize intracardiac structures in real-time. The steering controls allow clinicians to precisely navigate and position the catheter in the heart, ensuring optimal imaging and accessibility during diagnostic or interventional procedures. The catheter is inserted into a vein and advanced to the heart under imaging guidance, such as fluoroscopy. Steering controls are built into the catheter handle and usually consist of mechanical knobs, wheels, or levers. These controls enable bidirectional or multidirectional deflection of the catheter tip, allowing clinicians to maneuver it into specific cardiac chambers or target regions. ICE catheters may offer 3D steering capabilities, enhancing spatial orientation and providing comprehensive views of complex anatomical structures like valves, septa, or implanted devices.

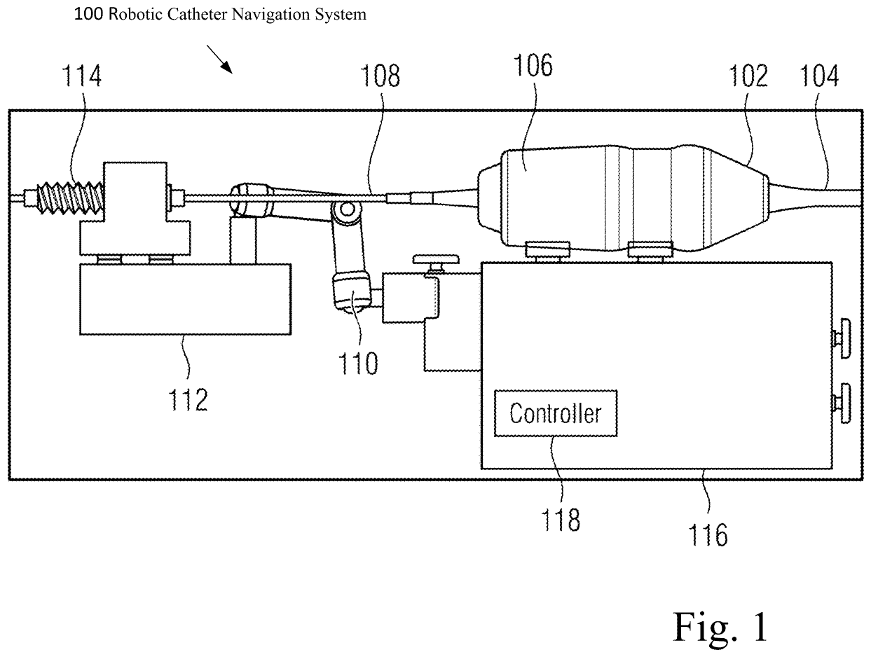

FIG. 1 depicts an example robotic catheter navigation system 100, in accordance with one or more embodiments. The robotic catheter navigation system 100 includes a catheter 108, a base 116, a catheter handle housing 102, an access point base 112, an access point guide 114, and an arm 110. In an embodiment, the catheter 108 is an ICE catheter for performing an ICE procedure, but may be any other suitable catheter. The base 116 and catheter handle housing 102 form a handle robot. The access point base 112 and access point guide 114 form an access point robot. The arm 110 connects or links the handle robot to the access point robot. The cable 104 interfaces with an ultrasound device (not shown) for, e.g., image processing, beam forming, displaying the generated image, etc. The depicted robotic catheter navigation system 100 of FIG. 1 is only one example. Other configurations of robotic catheter navigation system 100 are possible.

In the typically operation, a user manipulates (e.g., rotates) one or more rotatable knobs 106 to steer the catheter 108. In an embodiment, the knobs 106 include a first knob for bending the tip of catheter 108 in an anterior/posterior direction and a second knob for bending the tip of catheter 108 in a left/right direction. One or more motors (e.g., actuators) in base 116 drive gears in the handle robot to actuate movement (e.g., in the anterior/posterior direction and/or the left/right direction) of catheter 108 by applying pushing or pulling forces on steering wires (not shown) within catheter 108. The access point robot manipulates catheter 108 to provide more direct control of catheter 108 near the insertion point of the patient. The access point robot is configured to translate catheter 108 along its longitudinal axis and/or rotate catheter 108 about its longitudinal axis. Accordingly, robotic catheter navigation system 100 enables steering of catheter 108 is all four degrees of freedom anterior/posterior tip bending, left/right tip bending, rotation, and translation.

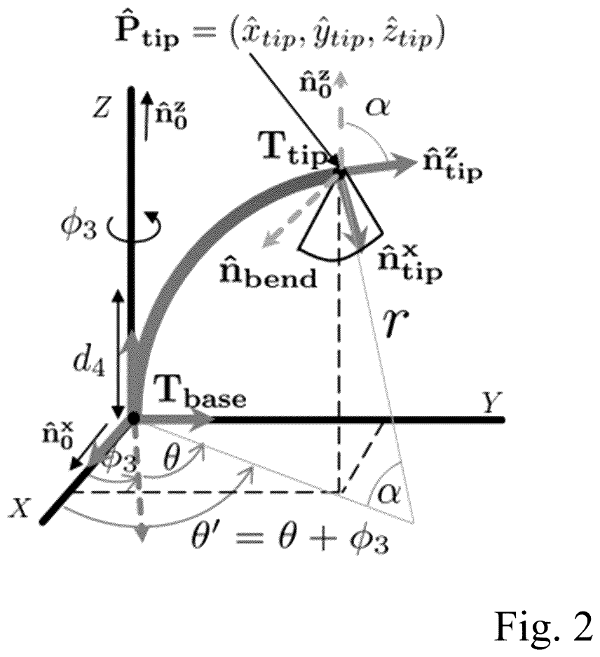

FIG. 2 shows the catheter bending geometry 500, in accordance with one or more embodiments. The term Tbase denotes the base coordinate frame for the bottom of the bending section of the catheter, Ttip denotes the catheter tip coordinate frame where the ultrasound array is installed, and N tip denotes the center of the image facing direction. Other exemplary robotic catheter navigation systems are described in U.S. patent application Ser. No. 16/809,067, filed Mar. 4, 2020, and U.S. patent application Ser. No. 16/907,675, filed Jun. 22, 2020, the disclosures of which are incorporated herein by reference in their entirety.

As multiple key positions are often required to observe the full extent of the patient's anatomy, complex navigation is required for the catheter from, for example the RA septal wall then through the tricuspid valve (TV) and through the right ventricle outflow tract (RVOT) and back to continue for an eventual ablation procedure. Current systems can create a three-dimensional echocardiographic image from multiple two-dimensional images, requiring the acquisition of multiple video clips via careful orientation of the probe, in most cases from the right atrium without the need for puncturing the transatrial septum. In this workflow, the operator is required to manually extract cardiac and respiratory gated frames from the clips, thus requiring the probe to be maintained in a specific position for a few heart cycles. The systems then leverage probe localization capabilities to obtain a 3D segmentation of the anatomy, either direct segmentation on 2D and projection in 3D or by the creation of an intermediate implicit sparse volume in 3D where the anatomy is then segmented. However depending on operator skill and image quality, navigation may be challenging and hinder this benefit. Furthermore, due to navigation constraints, the coverage of the anatomical model may be suboptimal and leave uncertainties in the overall appearance of the anatomy.

Embodiments solve these technical issues and others by providing an autonomous navigation system, guided by the ICE images that navigates between at least two key positions, for example, from RA septal wall, observing the LA left veins ostia and from RVOT observing the left atrial appendage (LAA). Two dimensional and three dimensional live catheter data may both be used without the need for a specific probe locating system. This allows for a close to real time scanning of the anatomy without manually holding the probe in specific positions. Additional advantages arise from the automated robotic control as some movements of the catheter 108 require precise simultaneous control of the multiple dials present on the catheter 108 which may not be achievable in a reproducible manner by a human operator. Additionally, the density of the anatomical map may then be precisely configured through the rotation increments to retain maximal anatomical fidelity and minimize the acquisition time.

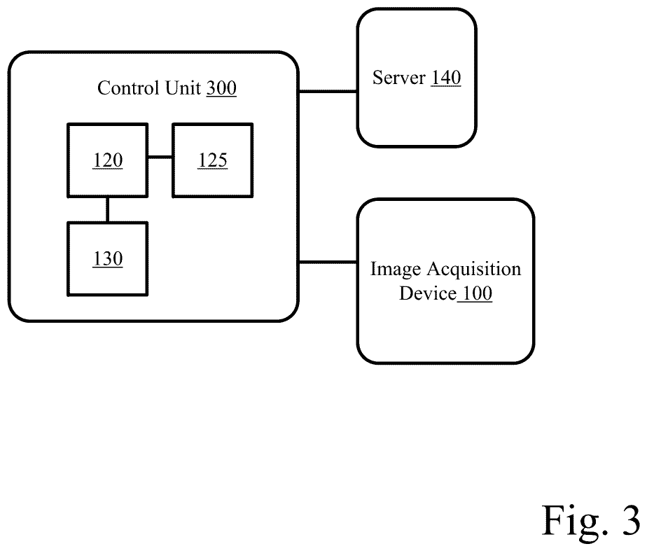

FIG. 3 depicts an example ultrasound system for autonomous navigation by an ICE catheter 108. The system includes an control unit 300, a robotic catheter navigation system 100 (also referred to as just an ICE catheter 108), and optionally a server 140. The server 140 may be configured to perform any of the tasks of the control unit 300 including processing and/or storing of the image data and models. The server 140 may be or include a cloud-based platform. The control unit 300 includes a processor 120, a memory 130, and a display 115. The control unit 300 may be included with or coupled with the robotic catheter navigation system 100. The control unit 300 is configured to acquire data using the robotic catheter navigation system 100. The control unit 300 is configured to automatically control the robotic catheter navigation system 100 as the catheter is maneuvered inside a patient's anatomy. The control unit 300 is configured to process acquired images from the robotic catheter navigation system 100 and generate a model of the anatomy of the patient. The model may be used for further control of the robotic catheter navigation system 100, analysis or diagnosis of the imaging data, and/or additional medical procedures.

The control unit 300 and/or server 140 may also be configured to train or store one or more machine learned models for these tasks. For example, the control unit 300 may be configured to train and/or store a deep reinforcement learning (DRL) agent 700 to control the catheter 108 using instructions based on acquired information from the robotic catheter navigation system 100. The control unit 300 includes a processor 120, a memory 130, and an interface 125. The control unit 300 may be configured to generate a simulated 3D environment from previously acquired image data to train the DRL agent 700. Additional, different, or fewer components may be provided. For example, a computer network is included for remote processing of locally captured image data, for example by the server 140. As another example, a user input device (e.g., keyboard, buttons, sliders, dials, trackball, mouse, or other device) is provided for user alteration or placement of one or more markers.

In an embodiment, the medical imaging device 130 is an ultrasound system 130 configured to generate 2D, 3D, and/or 4D ultrasound images of a patient. Ultrasound imaging uses sound waves to image internal body structures. The embodiments described herein use ICE as an example, but other ultrasound imaging techniques may be used. The example context of use is for treatment of atrial fibrillation under the guidance of ICE imaging. The left atrium may be the most common anatomy of interest for this context. The cardiac region includes the left atrial appendage (LAA), left inferior pulmonary vein (LIPV), left superior pulmonary vein (LSPV), right inferior pulmonary vein (RIPV), and right superior pulmonary vein (RSPV). Additional, different, or fewer structures or parts of interest may be included. Any of the structures may be portions, such as a root or base of a vein. The left ventricle or other heart chambers may be the anatomy of interest in other embodiments. Other ICE imaging locations may result in other anatomy of interest, such as arteries or veins. The left atrium and the corresponding anatomy are used as examples below. However, the automated control of the ICE catheter 108 may also be used for other procedures such as mitral valvuloplasty, transcatheter aortic valve replacement, and left atrial appendage closure among other procedures.

In an example, the ICE catheter 108 is an advanced intra-cardiac echocardiography (ICE) catheter that is configured to deliver high-resolution, real-time 4D volumetric imaging of intracardiac and pericardiac structures. The ICE catheter 108 integrates a phased array ultrasound transducer capable of producing 2D, 3D, and 4D volumetric imaging. The 4D (3D+time) capability allows for real-time visualization of dynamic cardiac structures, such as heart valves, septa, and blood flow patterns, providing critical spatial and temporal information. This comprehensive view may be used for guiding complex structural heart interventions, including transcatheter valve repair, atrial fibrillation ablation, and septal defect closures.

The ICE catheter 108 includes an ergonomic steering mechanism for example as described above in FIG. 1 that allows for precise control and maneuverability. The catheter's bidirectional or multidirectional steering allows for precise positioning of the catheter within the heart and provides an ability to navigate around anatomical landmarks. This flexibility is crucial for accessing hard-to-reach regions while ensuring optimal imaging angles. In order to provide precise control and maneuverability the ICE catheter 108 uses methods such as kinematics and non-linear elasticity compensation to accurately track the position and orientation of the ICE catheter tip. Non-linear elasticity compensation, for example, is a technique used to correct or account for the non-linear deformation behavior of materials under stress or load. In non-linear elasticity, the relationship between stress and strain is not proportional, causing deviations from predictable responses seen in linear elastic materials. Compensation involves incorporating mathematical models or control algorithms to predict and adjust for these non-linear effects.

In an embodiment, using a fixed coordinate system as a reference, the ICE catheter 108 represents the tip state as a combination of translation and rotation in three-dimensional space. This representation allows the ICE catheter 108 to interpret all of the catheter movements within the coordinate system. Consequently, the information from the ICE catheter 108 allows for the system to create a comprehensive map of all possible catheter tip positions, forming its configuration space. This space serves as a positional sensor, akin to electromagnetic (EM) sensors, providing precise information about the catheter's position for navigation and monitoring purposes, abet without requiring the additional hardware that is required for EM sensors, for example without the use of magnetic trackers.

In an embodiment, the processor 120 is configured to automatically control the movement of the ICE catheter 108 using a DRL agent 700. The processor 120 is a general processor, digital signal processor, graphics processing unit, application specific integrated circuit, field programmable gate array, artificial intelligence processor, digital circuit, analog circuit, combinations thereof, or other now known or later developed device for controlling the movement of a catheter, acquiring image data, and generating images and anatomy models, among other processes described below. The processor 120 is a single device, a plurality of devices, or a network. For more than one device, parallel or sequential division of processing may be used. Different devices making up the processor 120 may perform different functions. In one embodiment, the processor 120 is a control processor or other processor of the medical imaging device 130. In other embodiments, the processor 120 is part of a separate workstation or computer. The processor 120 operates pursuant to stored instructions to perform various acts described herein. The processor 120 is configured by software, design, firmware, and/or hardware to perform any or all of the acts of FIGS. 1, 4, and 5-8 and any other computations described herein.

The processor 120 is configured to train one or more machine learning networks. In an embodiment, a neural network of the DRL agent 700 is trained offline using reinforcement learning (RL). For RL, the DRL agent 700 interacts with a simulated environment to determine movement of a simulated catheter and, upon observing the consequences of its actions, learns to alter its own behavior in response to rewards received. The simulated environment 730/simulator may be an anatomical model generated from, for example, pre operative CT image data. The DRL agent 700 may be configured to observe a state S(t) from the simulator at timestep t. The agent 700 interacts with the simulator by taking an action A(t) in state S(t). When the agent 700 takes an action, the simulator and the agent 700 transition to a new state S(t+1) based on the current state 710 and the chosen action. The best sequence of actions is determined by the rewards provided by the simulator. Every time the simulator transitions to a new state 710, the simulator may also provide a reward 740 to the agent 700 as feedback. The goal of the agent 700 is to learn a policy (control strategy for operating the ICE catheter 108) that maximizes the expected return (cumulative, discounted reward 740).

In an embodiment, the agent 700 is positioned at a first position, moves to a second position, and obtains the current position information via acquired images or dead reckoning. At each time step, the agent 700 may receive a positive or negative reward 740 based on the current image data and/or positioning. The reward 740, (for example cumulative) guides the catheter toward the target position. The agent's state 710 is defined by the angular position of the catheter (e.g., catheter tip pose), the cartesian coordinates of the catheter, and location relative to the target position. The reward 740 provided by the simulator may be identified by the simulator or may be determined by an agent 700. The reward 740 may reflect efficient movement, data acquisition, or medical diagnostic results. The reward 740 may be based on multiple different values and may be determined using an algorithm that weigh different values differently. For example, the reward 740 may be calculated as a function of both a makespan and total cost (e.g. cost to run the machines). In an embodiment, Rewards are calculated at each commanded step, and based on negative reinforcement to encourage the agent 700 to move the catheter to the target position as efficiently as possible. This method allows negative rewards to accumulate with increasing magnitude if the catheter spends too many steps at larger distances from the target.

A Markov Decision Process (MDP) may be used to describe the agent 700 environment interaction, which includes a state space, an action space, a transition, a reward 740, and a discount factor. The state space is the set of possible states the agent can be in (for example position and orientation). Each state 710 represents a particular configuration or situation in the environment, for example, the catheter's pose and target. The action space is a set of possible actions that the agent 700 can take. Actions are the choices available to the agent 700 in each state 710. Actions can be discrete or continuous. The transition includes the probabilities that describe the dynamics of the environment. They specify the probability of transitioning from one state s to another state s′, if the agent 700 takes action a, which is represented as T(s′|s,a). It includes important information about the catheter/environment interaction, e.g., the interaction between the US probe and tissue. The reward 740 is the immediate feedback that the agent 700 receives from the environment for its action. It quantifies the desirability or value associated with transitions between states. states. The reward function may be designed to guide the agent 700 to achieve its goal. The discount factor is a value between 0 and 1 that determines the importance of future rewards compared to immediate rewards. It determines the preference of the agent 700 for immediate rewards or long term cumulative rewards. Given MDP(S,A,T,R,γ), the agent 700 chooses the action at state s with the observation o it receives according to the policy π(a|s). When the policy is deterministic, π is a mapping from state s to action a; when the policy is stochastic, π represents the possibility of selecting action a at state s. The goal of the RL is to find an optimal policy π* that maximizes the expectation of cumulative return.

The simulated environment 730 may be generated with pre-operative cardiac CT volumes and/or other imaging data. The simulated environment 730 may be an anatomical model of the cardiac region (or other regions). Key positions and anatomical landmarks are annotated in the CT volume and used to train the agent 700. For training, the agent 700 is placed virtually in selected key positions and attempts to navigate within the CT volume to target positions. Along the navigation pathway, synthetic ICE images may be generated from the CT local views via image synthesis deep learning algorithms. The synthetic ICE images may be used as feedback for calculating a state 710 or reward 740. The annotated landmarks from CT are also transferred to the generated ICE images. Thus, the agent 700 may be trained to recognize these landmarks from the ICE domain. The use of anatomical landmarks common across the patient population allows the definition of a standard coordinate system, in which the specific patient anatomy can be specified. This further allows definition of preset standard views (for example, home view, RVOT view etc.), and procedure specific navigation plans. The DRL agent 700 can then further refine the preset plans. Different neural networks may be used by the agent 700 for learning. As described above, one possibility is a deep reinforcement learning (DRL) network. The DRL network encodes high-dimensional state variables from the simulator into low-dimensional features to identify the high reward steps. The image processor 120 may be configured to generate the simulated environment 730 using one or more trained neural networks.

During a medical procedure, the processor 120 is configured to generate a model of the patient's anatomy from the image data acquired by the ICE catheter 108. The model may be used for diagnostic purposes or to assist in the medical procedure. The agent 700 is configured to maneuver the catheter though the patient's anatomy while acquiring image data using the catheter. The acquired data is sparse due to the incomplete sampling of all voxel locations. Due to the movement of the transducer and/or heart or other patient motion, less than all the volume is sampled during the ICE imaging. Some fields of view may include only one or less than all the structures of interest. Once assembled as a volume, less than all voxels may be represented by the ultrasound data. A segmentation method uses the sparse volume for better anatomical understanding and structural consistency as compared to a single two dimensional ICE image. Due to the sparsity and noisy nature of ultrasound data, segmentation using just the sparse ICE volume may not be as accurate as desired for diagnosis and/or guiding ablation. The sparse volume data may be input into a deep learning volumetric segmentation algorithm to obtain the contours of anatomical structures.

In an embodiment, an ICE volume is formed from the ultrasound data. The known locations represented by the ultrasound data are used to populate a volume. The positioning data from the ICE catheter 108 may be used to assign scalar or other ultrasound data to different voxels. Alternatively, the sensed positions are used to align the planar locations represented by the ultrasound data within the volume. The ultrasound data is mapped to three dimensions using the positions of the scan planes. The processor 120 may also provide real-time local updates on the anatomical map. During the intervention, for the part of the anatomical map that falls within the field of view of the robot catheter, the system may facilitate deep learning algorithms to perform live tracking of the structures and thus can overlay markers dynamically on the treatment region. This enables precision device delivery of the catheter and medical devices.

In an embodiment, the segmentation for generating the model, identifying landmarks, and key positions uses a machine-learned network. The acquired ICE data with or without other data are input to the machine-learned network and a segmentation/complete volume including key positions/landmarks may be output.

The machine-learned network may be an image-to-image network, such as a generator of a generative adversarial network (GAN), trained to convert the ICE volume to the complete volume and the 3D segmentation. For example, the trained convolution units, weights, links, and/or other characteristics of the network are applied to the data of the ICE volume and/or derived feature values to extract the corresponding features through a plurality of layers and output the complete volume and the 3D segmentation. The features of the input are extracted from the ICE images as arranged in 3D. Other more abstract features may be extracted from those extracted features using the architecture. Depending on the number and/or arrangement of units or layers, other features are extracted from the input.

Any machine training architecture for outputting a spatial distribution from an input spatial distribution may be used. For example, U-Net is used. A convolutional-to-transposed-convolutional network is used. One segment of layers or units applies convolution to increase abstractness or compression. The most abstract feature values are then output to another segment. The other segment of layers or units then applies transposed-convolution to decrease abstractness or compression, resulting in outputting of an indication of class membership by location. The architecture may be a fully convolutional network. A GAN includes a generator, such as the image-to-image or U-Net, and one or more discriminators. The generator includes an encoder (convolutional) network and decoder (transposed-convolutional) network forming a “U” shape with a connection between passing features at a greatest level of compression or abstractness from the encoder to the decoder. Skip connections may be provided. Any now known or later developed U-Net architectures may be used. Other fully convolutional networks may be used.

Image data, the machine trained networks, training data, and other data may be stored in the memory 130. The memory 130 may be or include an external storage device, RAM, ROM, database, and/or a local memory (e.g., solid state drive or hard drive). The same or different non-transitory computer readable media may be used for the instructions and other data. The memory 130 may be implemented using a database management system (DBMS) and residing on a memory 130, such as a hard disk, RAM, or removable media. Alternatively, the memory 130 is internal to the processor 120 (e.g., cache). The instructions for implementing the processes, methods, and/or techniques discussed herein are provided on non-transitory computer-readable storage media or memories, such as a cache, buffer, RAM, removable media, hard drive, or other computer readable storage media (e.g., the memory 130). The instructions are executable by the processor 120 or another processor. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor 120 or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

The display 115 may be configured to display or otherwise provide the images to the user. The display 115 is a CRT, LCD, projector, plasma, printer, tablet, smart phone or other now known or later developed display device for displaying the output.

In the existing protocol, using ICE is an opportunity for time saving during the ablation procedure, however depending on operator skill and image quality, navigation may be challenging and hinder this benefit. Furthermore, due to navigation constraints, the coverage of the anatomical model may be suboptimal and leave uncertainties in the overall appearance of the anatomy, to be eluded by the following ablation procedure. In certain embodiments, two dimensional and three dimensional live catheter data can both be used without the need for a specific probe locating system. This allows for a close to real time scanning of the anatomy without manually holding the probe in specific positions. Additional advantages arise from robotic control as some movements of the catheter require precise simultaneous control of the multiple dials present on the catheter which may not be achievable in a reproducible manner by a human operator. Additionally, the density of the anatomical map can then be precisely configured through the rotation increments to retain maximal anatomical fidelity and minimize the acquisition time.

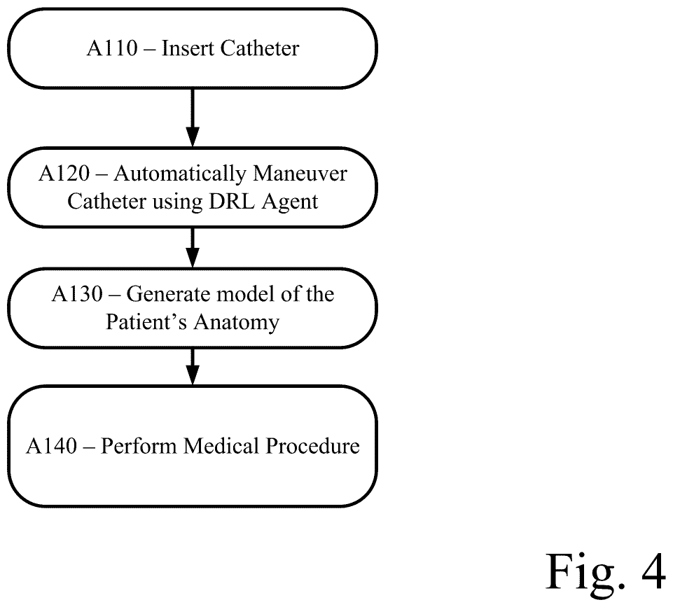

FIG. 4 depicts a method for generating an automated overview of the left atrial anatomy. The acts are performed by the system of FIGS. 1, 3, 6-8, other systems, a workstation, a computer, and/or a server. Additional, different, or fewer acts may be provided. The acts are performed in the order shown (e.g., top to bottom) or other orders. Certain acts may be omitted or changed depending on the results of the previous acts and the status of the patient. In an embodiment, the method is performed by a medical diagnostic ultrasound scanner with one or more ablation systems. An ICE catheter 108 is configured to acquire image data while being maneuvered through a patient's anatomy. A controller of the catheter automatically maneuvers the catheter based on actions provided by a machine trained agent 700. The ablation system, such as a power source connected with an electrode in the catheter or another catheter, performs may be used to perform a medical procedure based on the live anatomical mapping the cardiac anatomy. Other devices may be used to perform any of the acts.

At act A110, an ICE catheter 108 is inserted into a patient, for example at a first position. Catheter steering controls of the ICE catheter 108 are controlled at least in part by a machine trained agent 700. The machine trained agent 700 is configured to select the next best action based on the current state 710 of the ICE catheter 108 and its relationship with the end goal (for example, moving the ICE catheter 108 to a target position or acquiring the required image data). The current state 710 of the ICE catheter 108 may include the position and orientation of the ICE catheter 108. The current state 710 may be determined, for example, by reviewing past movements and current and past image data acquired by the ICE catheter 108. As the catheter progresses through the patient, a model may be generated with landmarks and key positions from the acquired image data. The model, as described below, may provide feedback for the agent 700 in the form of observations/states. The machine trained agent 700 is trained prior to implementation to determine which movements result in the best outcome. In an embodiment, the machine trained agent 700 is trained using deep reinforcement learning (DRL).

In DRL the machine trained agent 700 is generated and trained to self-develop an optimized method for efficiently moving from one position to another. The agent 700 may be trained to identify its current state 710 and determine which movements lead to an optimal outcome. For training, the agent 700 is introduced into a simulated environment 730, for example created by using preoperative CT images of multiple patients. The agent 700 is then allowed to maneuver the ICE catheter 108. If the agent 700 performs a “good” movement, the agent 700 is rewarded. If the agent 700 performs a “bad” movement, the agent 700 may be punished. Gradually, the agent 700 learns a policy during training to optimize the expected reward value of its actions. Expected rewards are determined by the reward value of the possible actions available to the agent 700 at time with the goal of moving the catheter from the first position to the second position (via maximizing expected reward value). Actions define the positional movement that occurs during state space transitions with respect to the state space's proximity to the target. Sequential actions may be determined and stored by the agent 700. The rewards may be derived from object detection, segmentation, tracking, and/or image registration. In order to learn optimal action policy in a sequence of learning episodes, the agent 700 is given random start-states and provided an environment that may be different from past attempts. During the exploration/training, periodic updates may be applied to the parameters of the neural network of the agent 700, leading to a more accurate approximation of the optimal Q function, given the current experience. This process is repeated in an iterative manner.

In an embodiment, clinician knowledge may be incorporated in the training and execution of the AI agent 700 employed for navigating the catheter using, for example, deep Q-learning from demonstrations. The clinician knowledge may include knowledge about the anatomy and common practice in path planning during specific procedures. An example of clinician knowledge may include that a typically ICE study starts at a home view obtained with a catheter placed in the mid-right atrium and the transducer in a neutral position facing the tricuspid valve. The home view provides imaging of the right atrium, tricuspid valve, right ventricle, and typically an oblique or short-axis view of the aortic valve. From the home view, clockwise rotation of the catheter brings into view the aortic valve in the long axis and the right ventricle outflow tract. Additional clockwise rotation allows visualization of the mitral valve and interatrial septum, with the left atrial appendage anteriorly and the coronary sinus posteriorly.

Once trained, the agent 700 may be used during a procedure. The agent 700 may be retrained and refined over time as feedback and additional training data is made available.

At act A120, the ICE catheter 108 is automatically maneuvered from a first position in a patient's anatomy to a second position in the patient's anatomy while acquiring image data of the patient's anatomy. The agent 700 generates instructions which are implemented by the ICE catheter 108 steering controls. As the ICE catheter 108 moves through the anatomy of the patient, the transducer of the ICE catheter 108 scans a plane. The scan plane is oriented based on a position of the catheter. As the catheter moves (e.g., translates or rotates), different scan planes are scanned. Each scan generates a frame of data representing the scan plane at that time. The frame of ultrasound data may be scalar values or display values (e.g., RGB) in a polar coordinate or Cartesian coordinate format. The frame of ultrasound data may be a B-mode, color flow, or other ultrasound image. A standard LA based coordinate system may be used that allows for a definition of standard views, navigation plans etc.

During navigation of the catheter, the current view of the catheter may be automatically classified as being a certain standard anatomical view (e.g., PLAX, PSAX, A4C, A3C, A2C, subcostal, or SSN) and anatomical objects of interest (e.g., the atriums, ventricles, pulmonary veins, left atrial appendages, etc.) shown in the current view of the catheter may be identified and the classification of the current view and the identified anatomical objects of interest may be identified in the current view. In an embodiment, the classification of the current view and the labelling of the anatomical objects of interest may be performed using a machine learning based network. The classification of the current view and the labelling of the anatomical objects of interest facilitate the navigation of the catheter by recognizing anatomical objects of interest encountered during the navigation of the catheter and providing the recognized anatomical objects of interest to the AI agent 700 for optimal planning of the next action.

This feedback may include these landmarks and other features which are identified in the acquired image data. Once the key positions are found, the robotic control of the catheter may be used to acquire 4D (3D over time) image clips of the anatomy while discretely rotating the catheter with a specific increment to observe its full extent. Moreover, the robotic control and imaging feedback system may be used for image stabilization purposes to limit misalignment issues. An efficient protocol may be used to minimize the acquisition time while maximizing the coverage. Automatic cardiac and respiratory gating may be performed directly from the images to extract the key frames in which the anatomy is in the same phase using deep learning algorithms. As the rotation was performed precisely by robotic control, the relative position of the obtained frames is known, allowing to fuse the obtained frames into a pseudo-volume through a registration process. The pseudo-volumes obtained at the distinct key locations described above, can also be registered together using local coordinates system that can be built from anatomical landmarks visible from both locations. As an example, the ostia of the left veins may be used as anchor point.

At act A130, a model of the patient's anatomy is generated from the acquired image data. As the catheter is maneuvered though the patient's anatomy, multiple images/planes are acquired representing a pseudo volume of the patient's anatomy. A sequence of frames of data result from the ICE imaging. Each frame represents a 2D scan plane, so a collection of frames representing different 2D scan planes in the volume of and/or around the heart are acquired. The processor 120 may generate a volumetric/3D segmentation of the patient's anatomy from input of the ICE volume to a machine-learned network. The anatomy represented by each location (voxel) is labeled. Alternatively, the segmentation forms a 3D mesh for each anatomy. Other segmentation results may be provided. The 3D segmentation provides a boundary/contour for one or more structures in 3D. The segmentation is of one or more structures of interest, such as identifying the locations of a sub-set or all the anatomical structures of interest discussed above for FIG. 2 for the left atrium. An annotated volumetric segmentation may provide an accurate model of the patient's anatomy that can be used during an intervention. The model may be also used as feedback for the agent 700 in addition to assisting in performing the medical procedure.

Different methods may be used for the segmentation and/or classification of the acquired image slice. For example, segmentation may be thresholding-based, region-based, shape-based, model based, neighboring based, and/or machine learning-based among other segmentation techniques. Thresholding-based methods segment the image data by creating binary partitions based on image attenuation values, as determined by the relative attenuation of structures on the images. Region-based segmentation compares one pixel in an image to neighboring pixels, and if a predefined region criterion (e.g., homogeneity) is met, then the pixel is assigned to the same class as one or more of its neighbors. Shape-based techniques use either an atlas-based approach or a model-based approach to find a boundary of the organ. Model-based methods use prior shape information, similar to atlas-based approaches; however, to better accommodate the shape variabilities, the model-based approaches may fit either statistical shape or appearance models of the organ to the image by using an optimization procedure. Neighboring anatomy-guided methods use the spatial context of neighboring anatomic objects. In machine learning-based methods, boundaries are predicted on the basis of the features extracted from the image data. A SoftMax layer or other classification technique, for example, may be provided to classify/identify the segmented pixels/boundaries/tissues etc.

The 3D segmentation may use a machine-learned network. The ICE volume with or without other data are input to the machine-learned network and a 3D segmentation is output in response. In an embodiment, the machine-learned network is an image-to-image network, such as a generator of a generative adversarial network (GAN), trained to convert the sparse ICE volume to a complete volume and/or a 3D segmentation. For example, the trained convolution units, weights, links, and/or other characteristics of the network are applied to the data of the ICE volume and/or derived feature values to extract the corresponding features through a plurality of layers and output the complete volume and the 3D segmentation. The features of the input are extracted from the ICE images as arranged in 3D. Other more abstract features may be extracted from those extracted features using the architecture. Depending on the number and/or arrangement of units or layers, other features are extracted from the input.

Machine learning for image segmentation may be done by extracting a selection of features from input images. These features may include, for example, pixel gray levels, pixel locations, image moments, information about a pixel's neighborhood, etc. A vector of image features is then fed into a learned classifier which classifies each pixel of the image into a class. The parameters of the classifier are learned automatically by giving the classifier input images for which the ground truth classification results is known. The output of the model is then compared to the ground truth, and the parameters of the model are adjusted so that the model's output better matches the ground truth value. This procedure is repeated for a large amount of input images, so that the learned parameters generalize to new, unseen examples. Deep learning may also be used for segmentation (and other tasks described herein), for example using a neural network. Deep learning-based image segmentation may be done, for example, using a convolutional neural network (CNN). The convolutional neural network includes a layered structure where series of convolutions are performed on an input image. Kernels of the convolutions are learned during training. The convolution results are then combined using a learned statistical model that outputs a segmented image. The image segmentation method may be trained using synthetically generated views.

At act A140, the model of the patient's anatomy may be used as feedback for the control of the ICE catheter 108 and/or performing the medical procedure. For example, the model may be used by the agent 700 to determine a next move or rotation of the ICE catheter 108. If the model is missing certain information or data, the ICE catheter 108 may be moved or rotated to acquire the required data. Embodiments provide for a close to real time scanning of the anatomy without manually holding the probe in specific positions. The model may further be used to determine the position of the catheter without using additional positioning mechanisms such as EM sensors. The model may be dynamically updated via real-time structure tracking by the catheter. During the intervention, for the part of the anatomical map that falls within the field of view of the robot catheter, the system may facilitate deep learning algorithms to perform live tracking of the structures and thus can overlay markers dynamically on the treatment region. This enables precision device delivery.

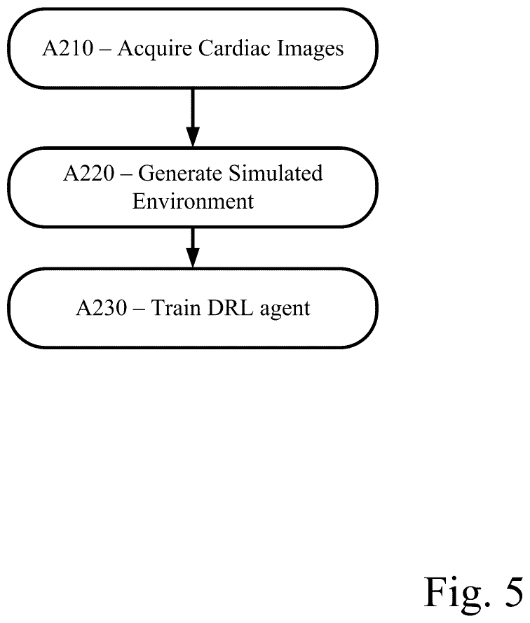

FIG. 5 depicts a method for training an agent 700 to operate a robotic ICE catheter 108. The acts are performed by the system of FIGS. 1, 3, 6-8, other systems, a workstation, a computer, and/or a server. Additional, different, or fewer acts may be provided. The acts are performed in the order shown (e.g., top to bottom) or other orders. Certain acts may be omitted or changed depending on the results of the previous acts and the status of the patient. In an embodiment, the method is performed using data acquired by a medical imaging scanner such as a CT scanner and/or a medical diagnostic ultrasound scanner with one or more ablation systems. The agent 700 is trained to control an ICE catheter 108 and corresponding transducer. The training occurs in a simulated environment 730 but may include feedback and/or data from actual procedures. The example provided below is for ICE and cardiac procedures but other imaging modalities and regions of a patient may be used.

At act A210, a plurality of cardiac images are acquired. In an embodiment, the cardiac images are pre-operative cardiac CT images. The cardiac CT images may be acquired at any point and may include images from multiple patients covering multiple different body types and features. In an embodiment multiple different environments may be generated below and as such different types of patient data may be acquired.

At act A220, one or more simulated cardiac environments 730 are generated from the plurality of cardiac CT volume. A simulated cardiac environment 730 may include multiple models of different patient and may include different features and abnormalities so that the agent 700 is trained using a comprehensive environment. The simulated cardiac environment 730 may further provide examples or estimates of image data that is acquired by the catheter at certain points. The image data may be used as feedback for determining the next best step by the agent 700 as described below.

At act A230, an agent 700 is trained to maneuver the ICE catheter 108 from a first position in the simulated cardiac environment 730 to a second position in the simulated cardiac environment 730 using deep reinforcement learning wherein rewards for the agent 700 are based on safety, efficiency, and best practices. The robotic catheter navigation system may include any robotic catheter navigation system. In an embodiment, the robotic catheter navigation system includes 12 degrees of freedom of the catheter. The 12 degrees of freedom of the catheter represents all possible actions for moving the catheter: positive and negative translation in the X, Y, Z plane and clockwise and counterclockwise rotation in the yaw, pitch, and roll axes. For training the agent 700, the next action is selected from the possible set of actions for moving the catheter.

The training of the agent 700 includes defining the set of actions, a set of rewards, and other parameters. The agent 700 is trained using DRL (Deep Reinforcement Learning) to continuously move closer to the target in each step. In one embodiment, a set of actions of the robotic navigation system is determined for navigating the catheter from the current view of the catheter towards the target based on a predicted path. The agent 700 may be trained to learn how to adjust its movements from the path based on acquired images of the anatomy as the agent 700 performs actions. For example, as each new patient has a different anatomy the agent 700 must learn the best next action given the current state 710 and current set of images that have been acquired.

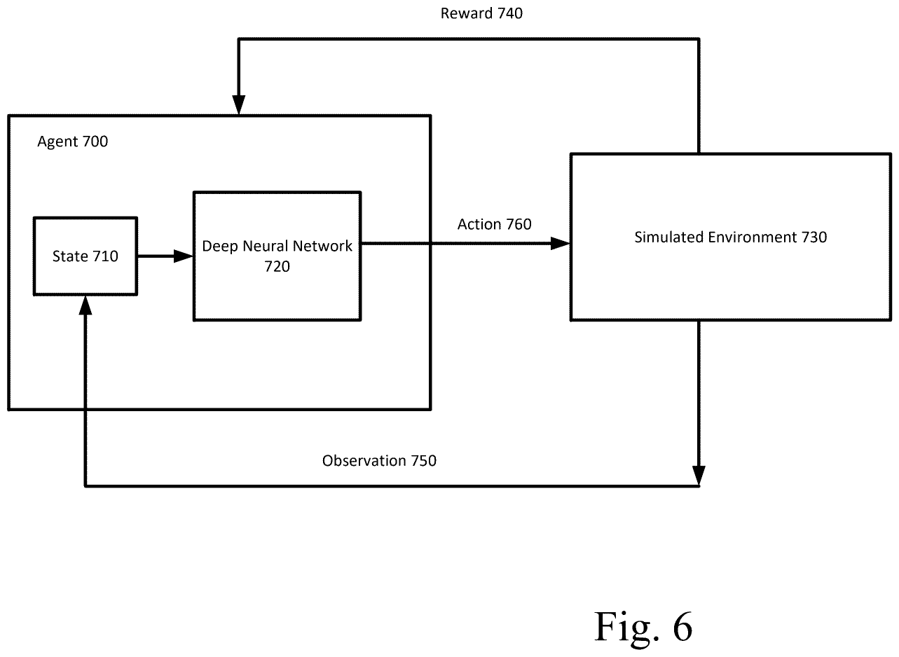

FIG. 6 depicts an example of a deep reinforcement learning process. An agent 700 learns to make decisions through trial and error. The agent 700 at every timestep is in a current state 710. The state 710 is input into the deep neural network (DNN) 720 which generates an action 760. The action 760 is input into the simulated environment 730 which provides a reward 740 and observation data 750 which are used to generate the next action 760 and state 710. The agent 700 attempts to learn a policy or map from observations to actions, in order to maximize its returns (expected sum of rewards). The learning may be performed using a neural network, e.g., the DNN 720. In an embodiment, the neural network may be a deep neural network, a convolutional neural network, or a convolutional deep neural network. Furthermore, the neural network may be an adversarial network, a deep adversarial network and/or a generative adversarial network.

Any type of suitable network may be used for training the agent 700 and other processes described herein. The network(s) may be defined as a plurality of sequential feature units or layers. Sequential is used to indicate the general flow of output feature values from one layer to input to a next layer. The information from the next layer is fed to a next layer, and so on until the final output. The layers may only feed forward or may be bidirectional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction the input data. The features are learned to reconstruct lower level features (i.e., features at a more abstract or compressed level). For example, features for generating a fused image or higher resolution image are learned. For a next unit, features for reconstructing the features of the previous unit are learned, providing more abstraction. Each node of the unit represents a feature. Different units are provided for learning different features.

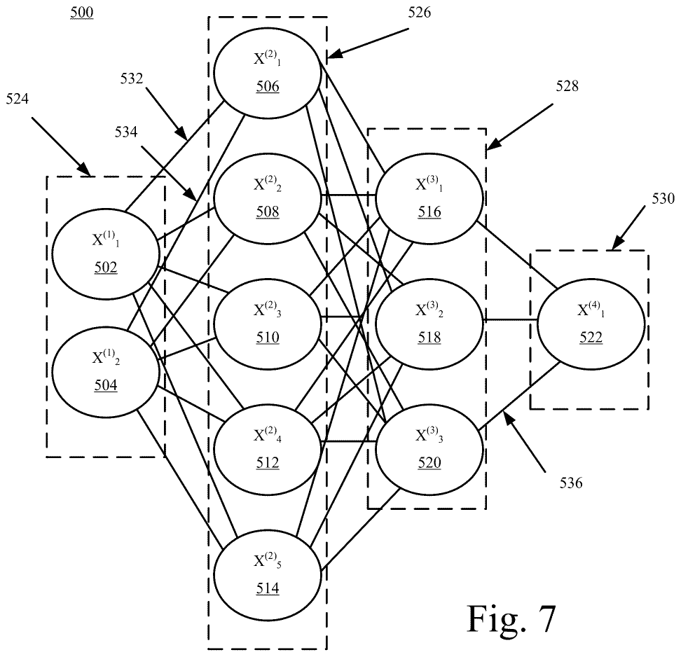

Various units or layers may be used, such as convolutional, pooling (e.g., max-pooling), deconvolutional, fully connected, or other types of layers. Within a unit or layer, any number of nodes is provided. For example, 100 nodes are provided. Later or subsequent units may have more, fewer, or the same number of nodes. In general, for convolution, subsequent units have more abstraction. FIG. 7 depicts an embodiment of an artificial neural network (ANN) 500, in accordance with one or more embodiments. Alternative terms for “artificial neural network” are “neural network”, “artificial neural net” or “neural net”. The artificial neural network 500 may be used in part in, for example, the one or more machine learning (deep learning) based networks utilized for training the agent 700.

The artificial neural network 500 includes nodes 502-522 and edges 532, 534, . . . , 536, wherein each edge 532, 534, . . . , 536 is a directed connection from a first node 502-522 to a second node 502-522. In general, the first node 502-522 and the second node 502-522 are different nodes 502-522, it is also possible that the first node 502-522 and the second node 502-522 are identical. For example, in FIG. 7, the edge 532 is a directed connection from the node 502 to the node 506, and the edge 534 is a directed connection from the node 504 to the node 506. An edge 532, 534, . . . , 536 from a first node 502-522 to a second node 502-522 is also denoted as “ingoing edge” for the second node 502-522 and as “outgoing edge” for the first node 502-522.

In this embodiment, the nodes 502-522 of the artificial neural network 500 may be arranged in layers 524-530, wherein the layers may include an intrinsic order introduced by the edges 532, 534, . . . , 536 between the nodes 502-522. In particular, edges 532, 534, . . . , 536 may exist only between neighboring layers of nodes. In the embodiment shown in FIG. 7, there is an input layer 524 including only nodes 502 and 504 without an incoming edge, an output layer 530 including only node 522 without outgoing edges, and hidden layers 526, 528 in-between the input layer 524 and the output layer 530. In general, the number of hidden layers 526, 528 may be chosen arbitrarily. The number of nodes 502 and 504 within the input layer 524 usually relates to the number of input values of the neural network 500, and the number of nodes 522 within the output layer 530 usually relates to the number of output values of the neural network 500.

In particular, a (real) number may be assigned as a value to every node 502-522 of the neural network 500. Here, x⁽ⁿ⁾ᵢ denotes the value of the i-th node 502-522 of the n-th layer 524-530. The values of the nodes 502-522 of the input layer 524 are equivalent to the input values of the neural network 500, the value of the node 522 of the output layer 530 is equivalent to the output value of the neural network 500. Furthermore, each edge 532, 534, . . . , 536 may include a weight being a real number, in particular, the weight is a real number within the interval [−1, 1] or within the interval [0, 1]. Here, w^{(m,n)}_{i,j} denotes the weight of the edge between the i-th node 502-522 of the m-th layer 524-530 and the j-th node 502-522 of the n-th layer 524-530. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 502-522 of the (n+1)-th layer 524-530 may be calculated based on the values of the nodes 502-522 of the n-th layer 524-530 by:

$${x}_{j}^{( n + 1 )} = f ( {\sum }_{i} {x}_{i}^{( n )} \cdot {w}_{I , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 524 are given by the input of the neural network 500, wherein values of the first hidden layer 526 may be calculated based on the values of the input layer 524 of the neural network, wherein values of the second hidden layer 528 may be calculated based in the values of the first hidden layer 526, etc.

In order to set the values w^{(m,n)}_{i,j} for the edges, the neural network 500 has to be trained using training data. In particular, training data includes training input data and training output data (denoted as tᵢ). For a training step, the neural network 500 is applied to the training input data to generate calculated output data. In particular, the training data and the calculated output data include a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ may be recursively calculated as

$${δ}_{j}^{( n )} = ( {\sum }_{k} {δ}_{k}^{( n + 1 )} \cdot {w}^{{( n )}_{i , j}} ) \cdot {f}^{′} ( {\sum }_{i} {x}^{{( n )}_{j}} \cdot {w}^{{( n )}_{i , j}} )$$

based on δ⁽ⁿ⁺¹⁾ⱼ, if the (n+1)-th layer is not the output layer, and

$${δ}_{j}^{( n )} = ( {x}^{{( n + 1 )}_{j}} - {t}^{{( n + 1 )}_{j}} ) \cdot {f}^{′} ( {\sum }_{i} {x}^{{( n )}_{j}} \cdot {w}^{{( n )}_{i , j}} )$$

if the (n+1)-th layer is the output layer 530, wherein f is the first derivative of the activation function, and t⁽ⁿ⁺¹⁾ⱼ is the comparison training value for the j-th node of the output layer 530.

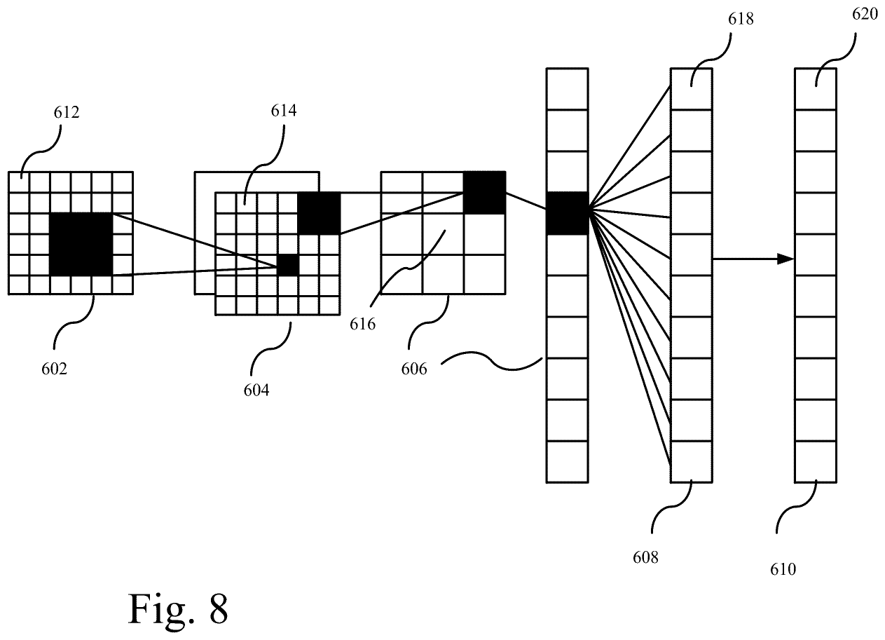

FIG. 8 shows an example convolutional neural network (CNN) 600, in accordance with one or more embodiments. Machine learning networks described herein, such as, e.g., the network 300 for training the agent 700, performing segmentation, etc. may be implemented using the convolutional neural network 600. In the embodiment shown in FIG. 8 the convolutional neural network includes 600 an input layer 602, a convolutional layer 604, a pooling layer 606, a fully connected layer 608, and an output layer 610. Alternatively, the convolutional neural network 600 may include several convolutional layers 604, several pooling layers 606, and several fully connected layers 608, as well as other types of layers. The order of the layers may be chosen arbitrarily, usually fully connected layers 608 are used as the last layers before the output layer 610.

In particular, within a convolutional neural network 600, the nodes 612-620 of one layer 602-610 may be considered to be arranged as a d-dimensional matrix or as a d-dimensional image. In particular, in the two-dimensional case the value of the node 612-620 indexed with i and j in the n-th layer 602-610 may be denoted as x⁽ⁿ⁾_{[i,j]}. However, the arrangement of the nodes 612-620 of one layer 602-610 does not have an effect on the calculations executed within the convolutional neural network 600 as such, since these are given solely by the structure and the weights of the edges.

In particular, a convolutional layer 604 is characterized by the structure and the weights of the incoming edges forming a convolution operation based on a certain number of kernels. In particular, the structure and the weights of the incoming edges are chosen such that the values x⁽ⁿ⁾ₖ of the nodes 614 of the convolutional layer 604 are calculated as a convolution x⁽ⁿ⁾ₖ=Kₖ*x⁽ⁿ⁻¹⁾ based on the values x⁽ⁿ⁻¹⁾ of the nodes 612 of the preceding layer 602, where the convolution * is defined in the two-dimensional case as:

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 612-620 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 612-620 in the respective layer 602-610. In particular, for a convolutional layer 604, the number of nodes 614 in the convolutional layer is equivalent to the number of nodes 612 in the preceding layer 602 multiplied with the number of kernels.

If the nodes 612 of the preceding layer 602 are arranged as a d-dimensional matrix, using a plurality of kernels may be interpreted as adding a further dimension (denoted as “depth” dimension), so that the nodes 614 of the convolutional layer 604 are arranged as a (d+1)-dimensional matrix. If the nodes 612 of the preceding layer 602 are already arranged as a (d+1)-dimensional matrix including a depth dimension, using a plurality of kernels may be interpreted as expanding along the depth dimension, so that the nodes 614 of the convolutional layer 604 are arranged also as a (d+1)-dimensional matrix, wherein the size of the (d+1)-dimensional matrix with respect to the depth dimension is by a factor of the number of kernels larger than in the preceding layer 602.

The advantage of using convolutional layers 604 is that spatially local correlation of the input data may exploited by enforcing a local connectivity pattern between nodes of adjacent layers, in particular by each node being connected to only a small region of the nodes of the preceding layer.

In embodiment shown in FIG. 8, the input layer 602 includes 36 nodes 612, arranged as a two-dimensional 6×6 matrix. The convolutional layer 604 includes 72 nodes 614, arranged as two two-dimensional 6×6 matrices, each of the two matrices being the result of a convolution of the values of the input layer with a kernel. Equivalently, the nodes 614 of the convolutional layer 604 may be interpreted as arranges as a three-dimensional 6×6×2 matrix, wherein the last dimension is the depth dimension.

A pooling layer 606 may be characterized by the structure and the weights of the incoming edges and the activation function of its nodes 616 forming a pooling operation based on a non-linear pooling function f. For example, in the two dimensional case the values x⁽ⁿ⁾ of the nodes 616 of the pooling layer 606 may be calculated based on the values x⁽ⁿ⁻¹⁾ of the nodes 614 of the preceding layer 604 as

$${x}^{( n )} [ i , j ] = f ( {x}^{( n - 1 )} [ {id}_{1} , {jd}_{2} ] , … , {x}^{( n - 1 )} [ {id}_{1} + {d}_{1} - 1 , {jd}_{2} + {d}_{2} - 1 ] )$$

In other words, by using a pooling layer 606, the number of nodes 614, 616 may be reduced, by replacing a number d1·d2 of neighboring nodes 614 in the preceding layer 604 with a single node 616 being calculated as a function of the values of said number of neighboring nodes in the pooling layer. In particular, the pooling function f may be the max-function, the average or the L2-Norm. In particular, for a pooling layer 606 the weights of the incoming edges are fixed and are not modified by training.

The advantage of using a pooling layer 606 is that the number of nodes 614, 616 and the number of parameters is reduced. This leads to the amount of computation in the network being reduced and to a control of overfitting.

In the embodiment shown in FIG. 8, the pooling layer 606 is a max-pooling, replacing four neighboring nodes with only one node, the value being the maximum of the values of the four neighboring nodes. The max-pooling is applied to each d-dimensional matrix of the previous layer.

A fully-connected layer 608 may be characterized by the fact that a majority, in particular, all edges between nodes 616 of the previous layer 606 and the nodes 618 of the fully-connected layer 608 are present, and wherein the weight of each of the edges may be adjusted individually.

In this embodiment, the nodes 616 of the preceding layer 606 of the fully-connected layer 608 are displayed both as two-dimensional matrices, and additionally as non-related nodes (indicated as a line of nodes, wherein the number of nodes was reduced for a better presentability). In this embodiment, the number of nodes 618 in the fully connected layer 608 is equal to the number of nodes 616 in the preceding layer 606. Alternatively, the number of nodes 616, 618 may differ.

A convolutional neural network 600 may also include a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks may be wired using summation (residual/dense neural networks), element-wise multiplication (attention) or other differentiable operators. Therefore, the convolutional neural network architecture may be nested rather than being sequential if the whole pipeline is differentiable.

In particular, convolutional neural networks 600 may be trained based on the backpropagation algorithm. For preventing overfitting, methods of regularization may be used, e.g. dropout of nodes 612-620, stochastic pooling, use of artificial data, weight decay based on the L1 or the L2 norm, or max norm constraints. Different loss functions may be combined for training the same neural network to reflect the joint training objectives. A subset of the neural network parameters may be excluded from optimization to retain the weights pretrained on another datasets.

Referring back to FIG. 5, at act A240, the trained agent 700 is output. The agent 700 may be used in a medical procedure to maneuver an ICE catheter 108 and acquire imaging data. The agent 700 may be retrained or updated as new data becomes available.

It is to be understood that the elements and features recited in the claims may be combined in different ways to produce new claims that likewise fall within the scope of the present invention. Thus, whereas the dependent claims below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description. Independent of the grammatical term usage, individuals with male, female or other gender identities are included within the term.

The following is a list of non-limiting illustrative embodiments disclosed herein:

Illustrative embodiment 1. A method for live anatomical mapping of a left atrium with robot assisted intra-cardiac echocardiography (ICE), the method comprising: inserting an ICE catheter into a patient, wherein catheter steering controls of the ICE catheter are controlled at least in part by a machine trained agent; automatically maneuvering the ICE catheter, using instructions from the machine trained agent, from a first position in a patient's anatomy to a second position in the patient's anatomy while acquiring image data of the patient's anatomy; and generating a model of the patient's anatomy from the acquired image data.

Illustrative embodiment 2. The method of embodiment 1, further comprising: tracking a position and orientation of an ICE catheter tip of the ICE catheter using the acquired image data of the patient's anatomy as the ICE catheter is maneuvered from the first position to the second position.

Illustrative embodiment 3. The method of embodiment 2, wherein the robot assisted ICE is performed without an additional positioning system.

Illustrative embodiment 4. The method of embodiment 1, wherein the machine trained agent is a Deep Reinforcement Learning (DRL) agent that interacts with the catheter steering controls, the DRL agent trained to move the ICE catheter to one or more key positions through a simulated environment.

Illustrative embodiment 5. The method of embodiment 4, wherein the simulated environment is constructed from a plurality of pre-operative cardiac CT volumes.

Illustrative embodiment 6. The method of embodiment 4, wherein the key positions and one or more anatomical landmarks are annotated in the simulated environment, wherein the machine trained agent is placed virtually in selected key positions and navigates within the simulated environment.

Illustrative embodiment 7. The method of embodiment 6, wherein along a navigation pathway, synthetic ICE images are generated from local views of the simulated environment.

Illustrative embodiment 8. The method of embodiment 1, wherein the ICE catheter is controlled with an external joystick providing a digital input directly mapped to one or more standard knob controls of the catheter.

Illustrative embodiment 9. The method of embodiment 1, wherein the automatically maneuvering of the ICE catheter is used for image stabilization to limit misalignment issues, automatic cardiac gating, and/or respiratory gating.

Illustrative embodiment 10. A method for training an agent to operate catheter steering controls of a ICE catheter, the method comprising: acquiring a plurality of pre-operative cardiac CT volumes; generating a simulated cardiac environment from the plurality of pre-operative cardiac CT volume; and training an agent to maneuver the ICE catheter from a first position in the simulated cardiac environment to a second position in the simulated cardiac environment using deep reinforcement learning.

Illustrative embodiment 11. The method of embodiment 10, further comprising: generating synthetic ICE images from CT local views of the simulated cardiac environment, wherein the synthetic ICE images are used in the deep reinforcement learning to define states, observations, and/or rewards.

Illustrative embodiment 12. The method of embodiment 10, wherein the agent is further trained to identify landmarks and key positions in the simulated cardiac environment.

Illustrative embodiment 13. The method of embodiment 10, wherein the catheter steering controls comprises twelve degrees of freedom of the catheter represents all possible actions for moving the catheter: positive and negative translation in a X, Y, Z plane and clockwise and counterclockwise rotation in a yaw, pitch, and roll axes.

Illustrative embodiment 14. The method of embodiment 10, wherein the first position comprises a right atrium septal wall observing a left atrium left veins ostia and the second position comprises a right ventricle outflow tract observing a left atrial appendage.

Illustrative embodiment 15. A system for live anatomical mapping of a left atrium of a patient with robot assisted intra-cardiac echocardiography (ICE), the system comprising: a machine trained agent, the machine trained agent trained using deep reinforcement learning using a simulated volumetric environment; and an ICE catheter comprising steering controls that are controlled at least in part by the machine trained agent; wherein the machine trained agent controls movement of the ICE catheter from a first position to a second position while acquiring imaging data of at least the left atrium.

Illustrative embodiment 16. The system of embodiment 15, further comprising: a processor configured to generate a model of the anatomy of the patient from the acquired imaging data.

Illustrative embodiment 17. The system of embodiment 16, wherein the model is used as feedback for controlling the movement of the ICE catheter.

Illustrative embodiment 18. The system of embodiment 16, wherein the processor is configured to track a position and orientation of an ICE catheter tip of the ICE catheter using the acquired image data of a patient's anatomy as the ICE catheter is maneuvered from the first position to the second position.

Illustrative embodiment 19. The system of embodiment 15, wherein the robot assisted ICE is performed without an additional positioning system.

Illustrative embodiment 20. The system of embodiment 15, wherein the first position comprises a right atrium septal wall observing a left atrium left veins ostia and the second position comprises a right ventricle outflow tract observing a left atrial appendage.

1. A method for live anatomical mapping of a left atrium with robot assisted intra-cardiac echocardiography (ICE), the method comprising:
inserting an ICE catheter into a patient, wherein catheter steering controls of the ICE catheter are controlled at least in part by a machine trained agent;
automatically maneuvering the ICE catheter, using instructions from the machine trained agent, from a first position in a patient's anatomy to a second position in the patient's anatomy while acquiring image data of the patient's anatomy; and
generating a model of the patient's anatomy from the acquired image data.

2. The method of claim 1, further comprising:
tracking a position and orientation of an ICE catheter tip of the ICE catheter using the acquired image data of the patient's anatomy as the ICE catheter is maneuvered from the first position to the second position.

3. The method of claim 2, wherein the robot assisted ICE is performed without an additional positioning system.

4. The method of claim 1, wherein the machine trained agent is a Deep Reinforcement Learning (DRL) agent that interacts with the catheter steering controls, the DRL agent trained to move the ICE catheter to one or more key positions through a simulated environment.

5. The method of claim 4, wherein the simulated environment is constructed from a plurality of pre-operative cardiac CT volumes.

6. The method of claim 4, wherein the key positions and one or more anatomical landmarks are annotated in the simulated environment, wherein the machine trained agent is placed virtually in selected key positions and navigates within the simulated environment.

7. The method of claim 6, wherein along a navigation pathway, synthetic ICE images are generated from local views of the simulated environment.

8. The method of claim 1, wherein the ICE catheter is controlled with an external joystick providing a digital input directly mapped to one or more standard knob controls of the catheter.

9. The method of claim 1, wherein the automatically maneuvering of the ICE catheter is used for image stabilization to limit misalignment issues, automatic cardiac gating, and/or respiratory gating.

10. A method for training an agent to operate catheter steering controls of a ICE catheter, the method comprising:
acquiring a plurality of pre-operative cardiac CT volumes;
generating a simulated cardiac environment from the plurality of pre-operative cardiac CT volume; and
training an agent to maneuver the ICE catheter from a first position in the simulated cardiac environment to a second position in the simulated cardiac environment using deep reinforcement learning.

11. The method of claim 10, further comprising:
generating synthetic ICE images from CT local views of the simulated cardiac environment, wherein the synthetic ICE images are used in the deep reinforcement learning to define states, observations, and/or rewards.

12. The method of claim 10, wherein the agent is further trained to identify landmarks and key positions in the simulated cardiac environment.

13. The method of claim 10, wherein the catheter steering controls comprises twelve degrees of freedom of the catheter represents all possible actions for moving the catheter: positive and negative translation in a X, Y, Z plane and clockwise and counterclockwise rotation in a yaw, pitch, and roll axes.

14. The method of claim 10, wherein the first position comprises a right atrium septal wall observing a left atrium left veins ostia and the second position comprises a right ventricle outflow tract observing a left atrial appendage.

15. A system for live anatomical mapping of a left atrium of a patient with robot assisted intra-cardiac echocardiography (ICE), the system comprising:
a machine trained agent, the machine trained agent trained using deep reinforcement learning using a simulated volumetric environment; and
an ICE catheter comprising steering controls that are controlled at least in part by the machine trained agent;
wherein the machine trained agent controls movement of the ICE catheter from a first position to a second position while acquiring imaging data of at least the left atrium.

16. The system of claim 15, further comprising:
a processor configured to generate a model of the anatomy of the patient from the acquired imaging data.

17. The system of claim 16, wherein the model is used as feedback for controlling the movement of the ICE catheter.

18. The system of claim 16, wherein the processor is configured to track a position and orientation of an ICE catheter tip of the ICE catheter using the acquired image data of a patient's anatomy as the ICE catheter is maneuvered from the first position to the second position.

19. The system of claim 15, wherein the robot assisted ICE is performed without an additional positioning system.

20. The system of claim 15, wherein the first position comprises a right atrium septal wall observing a left atrium left veins ostia and the second position comprises a right ventricle outflow tract observing a left atrial appendage.
